(19) United States
(12) Patent Application Publication
(10) Pub. No.: US 2011/0008283 A1
(43) Pub. Date: Jan. 13, 2011

(54) INTERFERON FUSION PROTEINS

(76) Inventors: Peter Artymiuk, Sheffield (GB); Richard Ross, Sheffield (GB); Jon Sayers, Chesterfield (GB)

Correspondence Address: KLARQUIST SPARKMAN, LLP, 121 SW SALMON STREET, SUITE 1600, PORTLAND, OR, 97204, US

(21) Appl. No.: 12/672,658

(22) PCT Filed: Aug. 5, 2008
(86) PCT No.: PCT/GB08/02638

§ 371 Date: Feb. 19, 2010

Related U.S. Application Data
(60) Provisional application No. 60/956,343, filed on Aug. 16, 2007.

(30) Foreign Application Priority Data
Aug. 8, 2007 (GB) 0715383.6

(51) Int. Cl. A61K 38/21; C07H 21/00; C07K 14/52; C12N 15/63; C12N 1/00; C12N 1/19; A61P 35/00

(52) U.S. Cl. 424/85.4; 536/23.52; 530/351; 435/320.1; 435/243; 435/254.2

(57) ABSTRACT

The disclosure relates to interferon fusion polypeptides and dimers; nucleic acid molecules encoding said polypeptides and methods of treatment that use said polypeptides/dimers.

The invention relates to interferon fusion polypeptides and dimers; nucleic acid molecules encoding said polypeptides and methods of treatment that use said polypeptides/dimers.

Cytokine receptors can be divided into two separate classes. Class 1 (referred to as the haematopoietic or growth hormone family) receptors are characterised by four conserved cysteine residues in the amino terminal part of their extracellular domain and the presence of a conserved Trp-Ser-Xaa-Trp-Ser motif in the C-terminal part. The receptors consist of two polypeptide chains. Class I receptors can be sub-divided into the GM-CSF sub-family (which includes IL-3, IL-5, GM-CSF, GCSF) and IL-6 sub-family (which includes IL-6, IL-11 and IL-12). In the IL-6 sub-family there is a common transducing subunit (gp130) that associates with one or two different cytokine subunits. There is a further sub-family referred to as the IL-2 sub-family (includes IL-2, IL-4, IL-7, IL-9 and IL-15. The repeated Cys motif is also present in Class 2 (interferon receptor family) the ligands of which are α, β and γ interferon but lack the conserved Trp-Ser-Xaa-Trp-Ser motif.

The interferons represent a generic group of cytokines and are classified into three groups; type I, type II and type III. Each of the interferon groups has an associated anti-viral and anti-proliferative activity and therefore recombinant forms are used to treat both viral infection and cancer.

Types 1 interferon includes interferon α, interferon β, interferon ε, interferon κ and ω interferon. Interferon α (IFNA), is produced predominantly by B lymphocytes but also by macrophages and can be sub-divided into 13 sub-types (IFNA 1, IFNA 2, IFNA 4, IFNA 5, IFNA 6, IFNA 7, IFNA 8, IFNA 10, IFNA 13, IFNA 14, IFNA 16, IFNA 17 and IFNA 21) and are found clustered on human chromosome 9. The level of homology between IFNA 1 isotypes is high being around 75-80% identity at the amino acid level. α interferon stimulates the activity of macrophages and Natural Killer (NK) cells to elicit an anti-viral response or an anti-tumour response. There is evidence that the combination of α interferon and β interferon results in the induction of the pro-apoptotic protein p53 and is thought to boost the p53 response to stress.

Type II interferon includes one member, interferon γ and is involved in the regulation of immune and inflammatory responses. In humans interferon γ is encoded by a single gene and is produced by activated T-cells and NK cells. Interferon γ does have anti-viral and anti-tumour activity however this is generally weaker when compared to interferon α. The function of interferon γ is to enhance the effects of Type I interferon by recruiting leukocytes to a site of infection and by stimulating macrophages to engulf invading bacteria during an infection. There is also an association of over-production of interferon γ and autoimmune disease.

Type III interferon includes three interferon A molecules referred to as IFN-λ1, IFN-λ2 and IFN-λ3. Type III interferon is also known to have anti-viral activity.

All human Type I interferon share a common cell surface receptor that comprises interferon Alpha receptor 1 (IFNAR1) and interferon Alpha receptor 2 (IFNAR2). Both these receptors belong to the class II cytokine receptor family. IFNAR2 is a high affinity binding component and can bind interferon in the absence of IFNAR1. IFNAR1 is recruited to the interferon IFNAR2 complex only after binding of IFNAR2 to a specific interferon. Structurally, IFNAR1 comprises three fibronectin domains linked via a transmembrane domain to a short cytoplasmic domain (100 amino acids). IFNAR2 differs from IFNAR1 in so far as it includes two fibronectin III domains and a longer cytoplasmic domain (250 amino acids). Type II interferon γ receptor comprises two subunits; IFN-γR1 is the ligand binding polypeptide and binds interferon γ and IFN-γR2 is the signal transducing polypeptide that activates the Janus kinases JAK 1 and JAK 2 during signal transduction. Type III interferon is unusual in so far as its members signal through a receptor complex comprising the IL10 receptor 2 and IFNLR1 receptor also referred to as interleukin 28 receptor.

The therapeutic activity of interferon has led to their development as recombinant protein in the control of viral infections and cancer. This disclosure relates to the identification of interferon recombinant forms that have improved pharmacokinetics and activity. The new interferon molecules are biologically active, form dimers and have improved stability.

According to an aspect of the invention there is provided a nucleic acid molecule comprising a nucleic acid sequence that encodes a polypeptide that has the activity of an interferon wherein said polypeptide comprises an interferon, or part thereof linked, directly or indirectly, to the interferon binding domain of an interferon receptor.

In a preferred embodiment of the invention said nucleic acid molecule encodes a polypeptide that has the activity of interferon α 2b wherein said polypeptide comprises interferon α 2b or part thereof linked, directly or indirectly, to the interferon binding domain of an interferon receptor.

According to an aspect of the invention there is provided a fusion polypeptide comprising: the amino acid sequence of an interferon, or active binding part thereof, linked, directly or indirectly, to the binding domain of an interferon receptor.

The invention includes type I, II and III interferon and isotypes thereof which include sequence variants.

An interferon sequence variant is a variant that varies from for example a reference polypeptide (e.g. IFNA1) and may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characters. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalaine, tyrosine and tryptophan. Most highly preferred are variants which retain the same biological function and activity as the reference polypeptide from which it varies.

A functionally equivalent polypeptide is a variant wherein one in which one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Ile; interchange of the hydroxyl residues Ser and Thr; exchange of the acidic residues Asp and Glu; substitution between amide residues Asn and Gln; exchange of the basic residues Lys and Arg; and replacements among aromatic residues Phe and Tyr.

In addition, the invention features polypeptide sequences having at least 75% identity with the polypeptide sequences illustrated in SEQ ID NO: 1 or 2, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated in SEQ ID NO: 1 or 2.

In a preferred embodiment of the invention said interferon is a type I interferon. Preferably said type I interferon is selected form the group consisting of: interferon α, interferon β, interferon ε, interferon κ and ω interferon

In a preferred embodiment of the invention said interferon α is selected from the group consisting of: IFNA 1, IFNA 2, IFNA 4, IFNA 5, IFNA 6, IFNA 7, IFNA 8, IFNA 10, IFNA 13, IFNA 14, IFNA 16, IFNA 17 and IFNA 21.

In a preferred embodiment of the invention said fusion polypeptide comprises SEQ ID NO: 1 or 2 or an amino acid sequence that is at least 75% identical to the amino acid sequence represented in SEQ ID NO: 1 or 2 over all or part of its length.

In a preferred embodiment of the invention said fusion polypeptide comprises SEQ ID NO: 1 or 2 or an amino acid sequence that is at least 80% identical to the amino acid sequence represented in SEQ ID NO: 1 or 2 over all or part of its length.

In a preferred embodiment of the invention said fusion polypeptide comprises SEQ ID NO: 1 or 2 or an amino acid sequence that is at least 85% identical to the amino acid sequence represented in SEQ ID NO: 1 or 2 over all or part of its length.

In a preferred embodiment of the invention said fusion polypeptide comprises SEQ ID NO: 1 or 2 or an amino acid sequence that is at least 90% identical to the amino acid sequence represented in SEQ ID NO: 1 or 2 over all or part of its length.

In a preferred embodiment of the invention said fusion polypeptide comprises SEQ ID NO: 1 or 2 or an amino acid sequence that is at least 95% identical to the amino acid sequence represented in SEQ ID NO: 1 or 2 over all or part of its length.

In a preferred embodiment of the invention said fusion polypeptide comprises SEQ ID NO: 1 or 2.

In a preferred embodiment of the invention there is provided a fusion polypeptide comprising: the amino acid sequence of an interferon α 2b or active binding part thereof, linked, directly or indirectly, to the binding domain of an interferon receptor.

In a preferred embodiment of the invention said fusion polypeptide comprises SEQ ID NO: 34 or 35 or an amino acid sequence that is at least 75%, 80%, 85%, 90% or 95% identical to the amino acid sequence represented in SEQ ID NO: 34 or 35 over all or part of its length.

In a preferred embodiment of the invention said fusion polypeptide comprises SEQ ID NO: 34 or 35.

In a preferred embodiment of the invention said fusion polypeptide comprises at least one, two or three fibronectin III binding domain.

In a preferred embodiment of the invention said fusion polypeptide comprises amino acid residues 28-436 of SEQ ID NO: 5.

In a preferred embodiment of the invention said fusion polypeptide comprises amino acid residues 27-243 of SEQ ID NO: 6.

In a preferred embodiment of the invention interferon α 2b is linked to an interferon binding domain of an interferon receptor wherein said interferon α 2b is positioned amino terminal to said binding domain in said fusion polypeptide.

In an alternative preferred embodiment of the invention interferon α 2b is linked to an interferon binding domain of an interferon receptor wherein said interferon α 2b is positioned carboxyl-terminal to said binding domain in said fusion polypeptide.

In a preferred embodiment of the invention said interferon α 2b is linked to the binding domain of the of the interferon receptor by a peptide linker.

Preferably said peptide linking molecule comprises at least 1, 2, 3, 4, 5 or 6 copies of the peptide Gly Gly Gly Gly Ser.

In a preferred embodiment of the invention said peptide linking molecule consists of 5 copies of the peptide Gly Gly Gly Gly Ser.

In an alternative preferred embodiment of the invention said polypeptide does not comprise a peptide linking molecule and is a direct fusion of interferon α 2b and the interferon binding domain of the interferon receptor.

In an alternative preferred embodiment of the invention said interferon is interferon β.

In a preferred embodiment of the invention said fusion polypeptide comprises SEQ ID NO: 3 or 4.

In a preferred embodiment of the invention there is provided a fusion polypeptide comprising at least one fibronectin III binding domain.

In a preferred embodiment of the invention there is provided a fusion polypeptide comprising or consisting of two fibronectin III binding domains.

In a preferred embodiment of the invention there is provided a fusion polypeptide comprising or consisting of three fibronectin III binding domains.

In a further preferred embodiment of the invention said fusion polypeptide comprises amino acid residues 28-436 of SEQ ID NO: 5.

In a further preferred embodiment of the invention said fusion polypeptide comprises amino acid residues 27-243 of SEQ ID NO: 6.

In an alternative preferred embodiment of the invention said interferon is a type II interferon. Preferably said type II interferon is interferon γ.

In a preferred embodiment of the invention interferon γ is represented by SEQ ID NO: 7 or 8.

In a preferred embodiment of the invention the interferon binding domain of an interferon receptor is an interferon γ receptor binding domain comprising SEQ ID NO: 9 or 10.

In a still further preferred embodiment of the invention said interferon is a type III interferon. Preferably said type III interferon is selected from the group consisting of IFN-λ1, IFN-λ2 and IFN-λ3.

In a preferred embodiment of the invention IFN-λ1 is represented by SEQ ID NO: 11 or 12.

In a preferred embodiment of the invention IFN-λ2 is represented by SEQ ID NO: 13 or 14.

In a preferred embodiment of the invention IFN-λ3 is represented by SEQ ID NO: 15 or 16.

In a preferred embodiment of the invention the interferon binding domain of an interferon receptor is an interferon λ receptor binding domain comprising SEQ ID NO: 17 or 18.

In a preferred embodiment of the invention the interferon binding domain of an interferon receptor is an interferon λ receptor binding domain comprising SEQ ID NO: 19 or 20.

In a preferred embodiment of the invention interferon is linked to an interferon binding domain of an interferon receptor wherein said interferon is positioned amino terminal to said binding domain in said fusion polypeptide.

In an alternative preferred embodiment of the invention interferon is linked to an interferon binding domain of an interferon receptor wherein said interferon is positioned carboxyl-terminal to said binding domain in said fusion polypeptide.

In a preferred embodiment of the invention said interferon is linked to the binding domain of the of the interferon receptor by a peptide linker; preferably a flexible peptide linker.

In a preferred embodiment of the invention said peptide linking molecule comprises at least one copy of the peptide Gly Gly Gly Gly Ser.

In a preferred embodiment of the invention said peptide linking molecule comprises 2, 3, 4, 5 or 6 copies of the peptide Gly Gly Gly Gly Ser.

Preferably said peptide linking molecule consists of 5 copies of the peptide Gly Gly Gly Gly Ser.

In a still further alternative embodiment of the invention said polypeptide does not comprise a peptide linking molecule and is a direct fusion of interferon and the interferon binding domain of the interferon receptor.

According to an aspect of the invention there is provided a nucleic acid molecule comprising a nucleic acid sequence selected from:
i) a nucleic acid sequence as represented in SEQ ID NO:21;
ii) a nucleic acid sequence as represented in SEQ ID NO:24;
iii) a nucleic acid sequence as represented in SEQ ID NO: 27;
iv) a nucleic acid sequence as represented in SEQ ID NO: 30; or

a nucleic acid molecule comprising a nucleic sequence that hybridizes under stringent hybridization conditions to SEQ ID NO 21, 24, 27, 30 or 33 and which encodes a polypeptide that has interferon modulating activity.

In a preferred embodiment of the invention said nucleic acid molecule encodes a polypeptide that has agonist activity.

In a preferred embodiment of the invention said nucleic acid molecule encodes a polypeptide that has antagonist activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (allows sequences that share at least
90% identity to hybridize)

| | |
|---|---|
| Hybridization: | 5x SSC at 65° C. for 16 hours |
| Wash twice: | 2x SSC at room temperature (RT) for 15 minutes |
| | each |
| Wash twice: | 0.5x SSC at 65° C. for 20 minutes each |

High Stringency (allows sequences that share at least
80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share at least
50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said nucleic acid molecule comprises or consists of a nucleic acid sequence as represented in SEQ ID NO: 21.

In a preferred embodiment of the invention said nucleic acid molecule comprises or consists of a nucleic acid sequence as represented in SEQ ID NO: 24.

In a preferred embodiment of the invention said nucleic acid molecule comprises or consists of a nucleic acid sequence as represented in SEQ ID NO: 27.

In a preferred embodiment of the invention said nucleic acid molecule comprises or consists of a nucleic acid sequence as represented in SEQ ID NO: 30.

In a preferred embodiment of the invention said nucleic acid molecule comprises or consists of the nucleic acid sequence in SEQ ID NO: 33.

According to an aspect of the invention there is provided a polypeptide encoded by the nucleic acid according to the invention.

According to a further aspect of the invention there is provided a polypeptide comprising or consisting of an amino acid sequence selected from the group consisting of: SEQ ID NO: 22, 23, 25, 26, 28, 29, 31, 32, 34 or 35.

According to an aspect of the invention there is provided a homodimer consisting of two polypeptides wherein each of said polypeptides comprises:
i) a first part comprising interferon, or a receptor binding domain thereof, optionally linked by a peptide linking molecule to
ii) a second part comprising at least one interferon binding domain or part thereof, of an interferon receptor.

In a preferred embodiment of the invention said homodimer comprises two polypeptides comprising or consisting of SEQ ID NO: 22, 23, 25, 26, 28, 29, 31, 32, 34 or 35.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection. Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to an aspect of the invention there is provided a vaccine composition comprising a nucleic acid molecule or polypeptide according to the invention and an antigenic molecule.

It will be apparent to the skilled artisan that the polypeptides of the invention are potent adjuvants. An adjuvant is a substance or procedure which augments specific immune responses to antigens by modulating the activity of immune cells. Examples of adjuvants include Freunds adjuvant, muramyl dipeptides or liposomes. An adjuvant is therefore an immunomodulator. The fusion polypeptides of the invention may be administered either as a polypeptide adjuvant or as a nucleic acid molecule in the example of DNA vaccination. Vaccine compositions may also include a carrier. Some polypeptide or peptide antigens contain B-cell epitopes but no T cell epitopes. Immune responses can be greatly enhanced by the inclusion of a T cell epitope in the polypeptide/peptide or by the conjugation of the polypeptide/peptide to an immunogenic “carrier” protein such as key hole limpet haemocyanin or tetanus toxoid which contain multiple T cell epitopes.

In a preferred embodiment of the invention said antigenic molecule is a viral polypeptide antigen.

Viral pathogens are a major source of disease in humans and animals, for example live stock animals. Viral antigens are derived from a viral pathogens such as Human Immunodeficiency Virus; Human T Cell Leukaemia Virus, Ebola virus or other haemorrhagic fever virus, human papilloma virus (HPV) that cause cervical cancer and other cancers, papovavirus, rhinovirus, poliovirus, herpesvirus, adenovirus, Epstein Barr virus, influenza virus A, B or C, Hepatitis B and C viruses, Variola virus, rotavirus or SARS coronavirus.

In an alternative preferred embodiment of the invention said antigenic molecule is a cancer antigen.

The term “antigenic molecule” refers to a nucleotide sequence, the expression of which in a target cell results in the production of a cell surface antigenic protein capable of recognition by the immune system. The antigenic molecule is derived from a tumour cell specific antigen; ideally a tumour rejection antigen. Tumour rejection antigens are well known in the art and include, for example, the MAGE, BAGE, GAGE and DAGE families of tumour rejection antigens, see Schulz et al Proc Natl Acad Sci USA, 1991, 88, pp 991-993. It has been known for many years that tumour cells produce a number of tumour cell specific antigens, some of which are presented at the tumour cell surface. These are generally referred to as tumour rejection antigens and are derived from larger polypeptides referred to as tumour rejection antigen precursors. Tumour rejection antigens are presented via HLA's to the immune system. The immune system recognises these molecules as foreign and naturally selects and destroys cells expressing these antigens. If a transformed cell escapes detection and becomes established a tumour develops. Vaccines have been developed based on dominant tumour rejection antigen's to provide individuals with a preformed defence to the establishment of a tumour.

According to a further aspect of the invention there is provided a method to treat a human subject suffering from a viral infection comprising administering an effective amount of a polypeptide according to the invention.

According to a further aspect of the invention there is provided a method to treat a human subject suffering from cancer comprising administering an effective amount of a polypeptide according to the invention.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumours of mesenchymal derivation.

In a preferred method of the invention said cancer is melanoma.

According to a further aspect of the invention there is provided a method to treat a human subject suffering from multiple sclerosis comprising administering an effective amount of a fusion polypeptide according to the invention.

In an alternative preferred method of the invention said fusion polypeptide comprises interferon β.

In a preferred method of the invention said fusion polypeptide comprises SEQ ID NO: 3 or 4.

In a further preferred embodiment of the invention said polypeptide is administered at two day intervals; preferably said polypeptide is administered at weekly, 2 weekly or monthly intervals.

According to an aspect of the invention there is provided the use of a polypeptide according to the invention for the manufacture of a medicament for the treatment of viral infection.

According to an aspect of the invention there is provided the use of a polypeptide according to the invention for the manufacture of a medicament for the treatment of cancer.

In a preferred embodiment of the invention said cancer is melanoma.

According to an aspect of the invention there is provided the use of a polypeptide according to the invention for the manufacture of a medicament for the treatment of multiple sclerosis.

In a preferred embodiment of the invention said fusion polypeptide comprises interferon β.

In a preferred embodiment of the invention said fusion polypeptide comprises SEQ ID NO: 3 or 4.

According to a further aspect of the invention there is provided a monoclonal antibody that binds the polypeptide or dimer according to the invention.

Preferably said monoclonal antibody is an antibody that binds the polypeptide or dimer but does not specifically bind interferon or interferon receptor individually.

The monoclonal antibody binds a conformational antigen presented either by the polypeptide of the invention or a dimer comprising the polypeptide of the invention.

In a further aspect of the invention there is provided a method for preparing a hybridoma cell-line producing monoclonal antibodies according to the invention comprising the steps of:
i) immunising an immunocompetent mammal with an immunogen comprising at least one polypeptide according to the invention;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

Preferably, the said immunocompetent mammal is a mouse. Alternatively, said immunocompetent mammal is a rat.

The production of monoclonal antibodies using hybridoma cells is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V.II ed. by Schwartz, 1981, which are incorporated by reference.

According to a further aspect of the invention there is provided a hybridoma cell-line obtained or obtainable by the method according to the invention.

According to a further aspect of the invention there is provided a diagnostic test to detect a polypeptide according to the invention in a biological sample comprising:
i) providing an isolated sample to be tested;
ii) contacting said sample with a ligand that binds the polypeptide or dimer according to the invention; and
iii) detecting the binding of said ligand in said sample.

In a preferred embodiment of the invention said ligand is an antibody; preferably a monoclonal antibody.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

Table 1 is a summary of LR nomenclature;

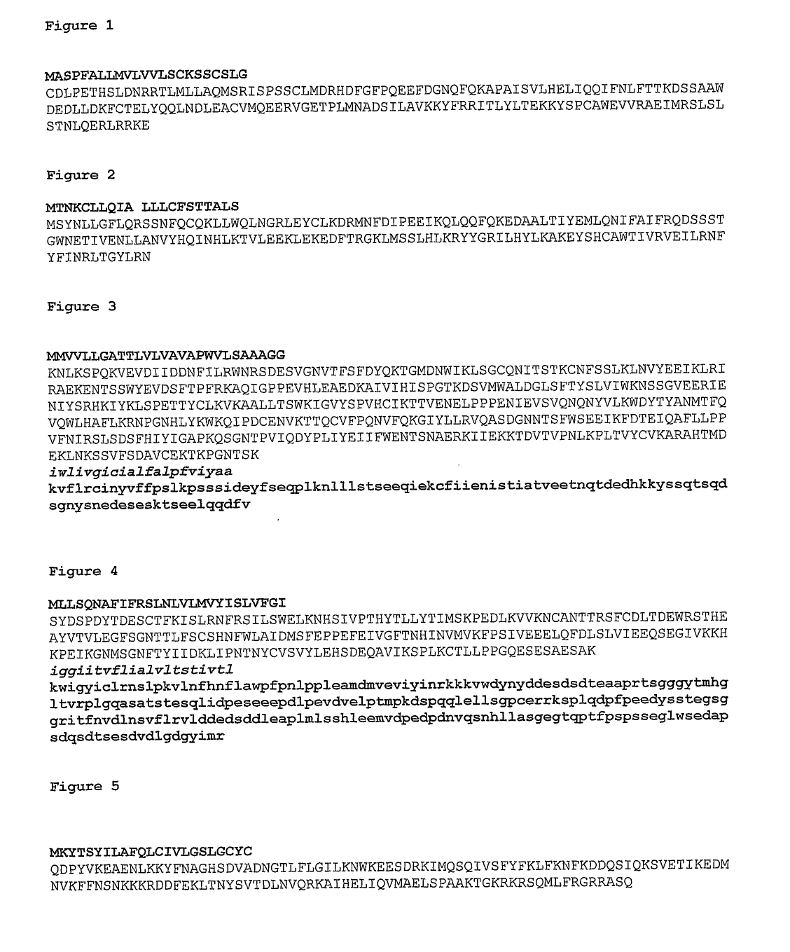

FIG. 1 is the amino acid sequence of human IFNA 1(signal sequence in bold capitals; mature protein in non-bold capitals);

FIG. 2 is the amino acid sequence of human interferon beta (signal sequence in bold capitals; mature protein in non-bold capitals);

FIG. 3 is the amino acid sequence of the human IFNR1 receptor (signal sequence in bold capitals (ss); extracellular domain of the mature protein in non-bold capitals; transmembrane domain in lower case italics; and cytoplasmic domain in bold lower case;

FIG. 4 is the amino acid sequence of the human IFNR2 receptor;

FIG. 5 is the amino acid sequence of interferon gamma;

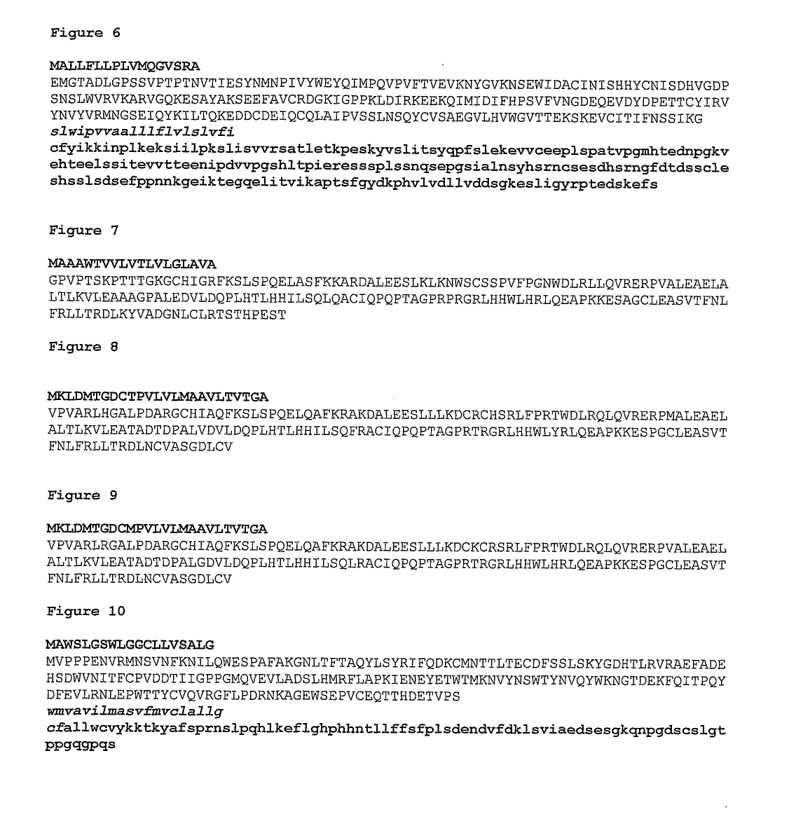

FIG. 6 is the amino acid sequence of interferon gamma receptor IFNgamma R1;

FIG. 7 is the amino acid sequence of interferon lambda 1;

FIG. 8 is the amino acid sequence of interferon lambda 2;

FIG. 9 is the amino acid sequence of interferon lambda 3;

FIG. 10 is the amino acid sequence of interferon lambda receptor IL10 receptor 2;

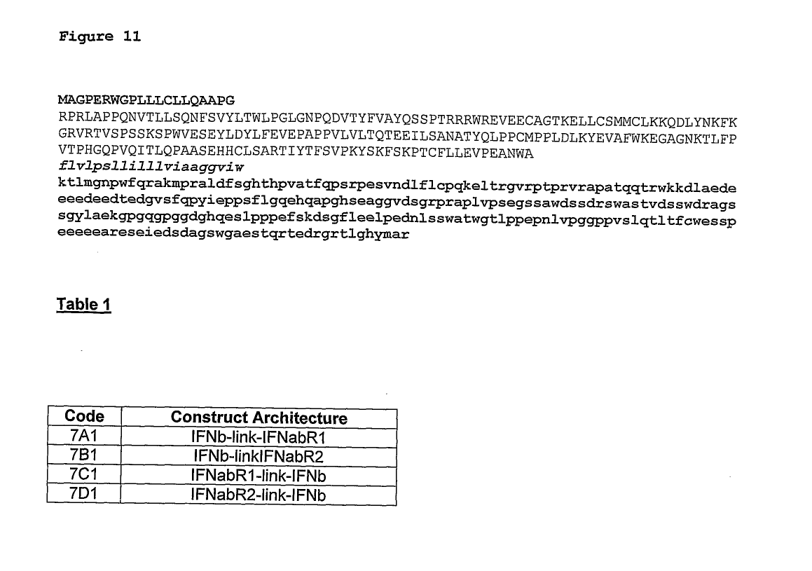

FIG. 11 is the amino acid sequence of interferon lambda receptor IFNLR1;

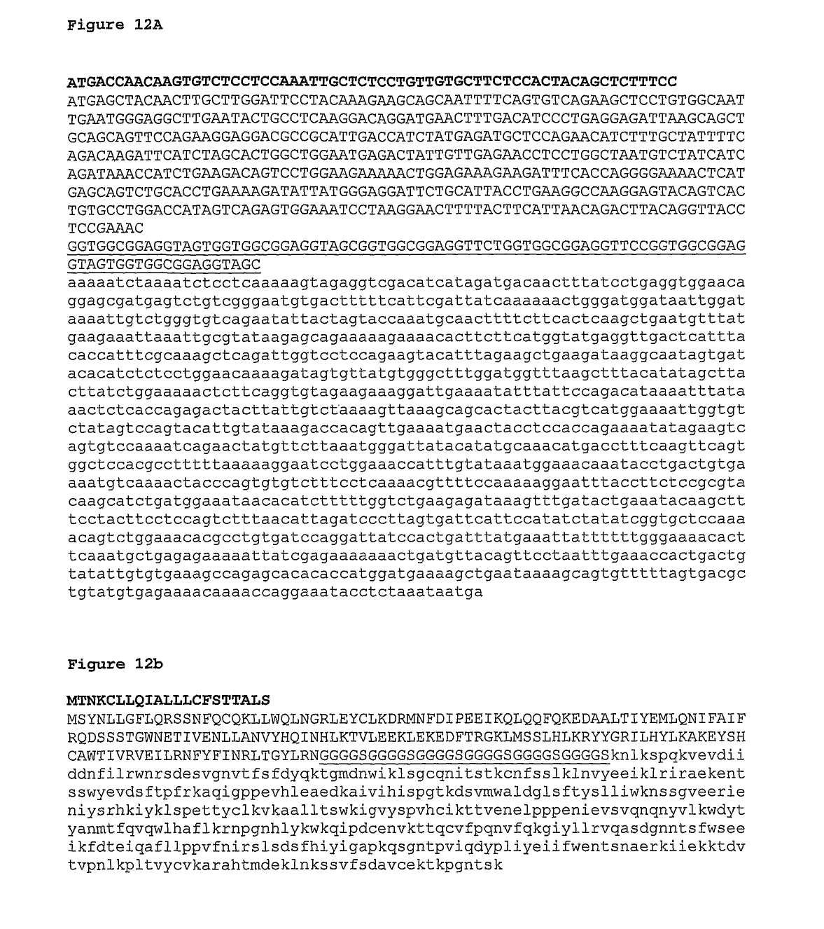

FIG. 12a is the nucleic acid sequence of LR 7A1; FIG. 12b is the amino acid sequence of LR 7A1;

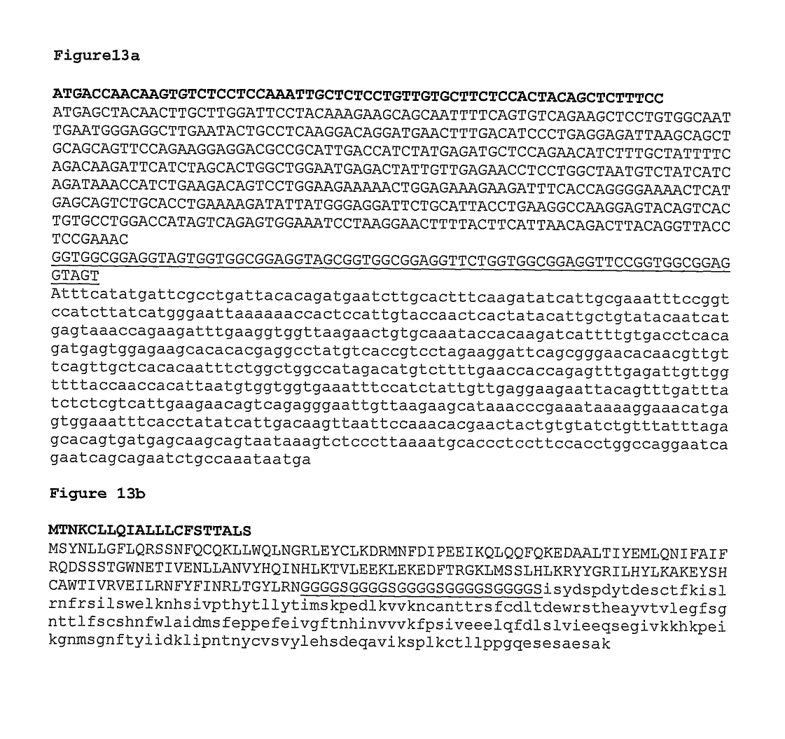

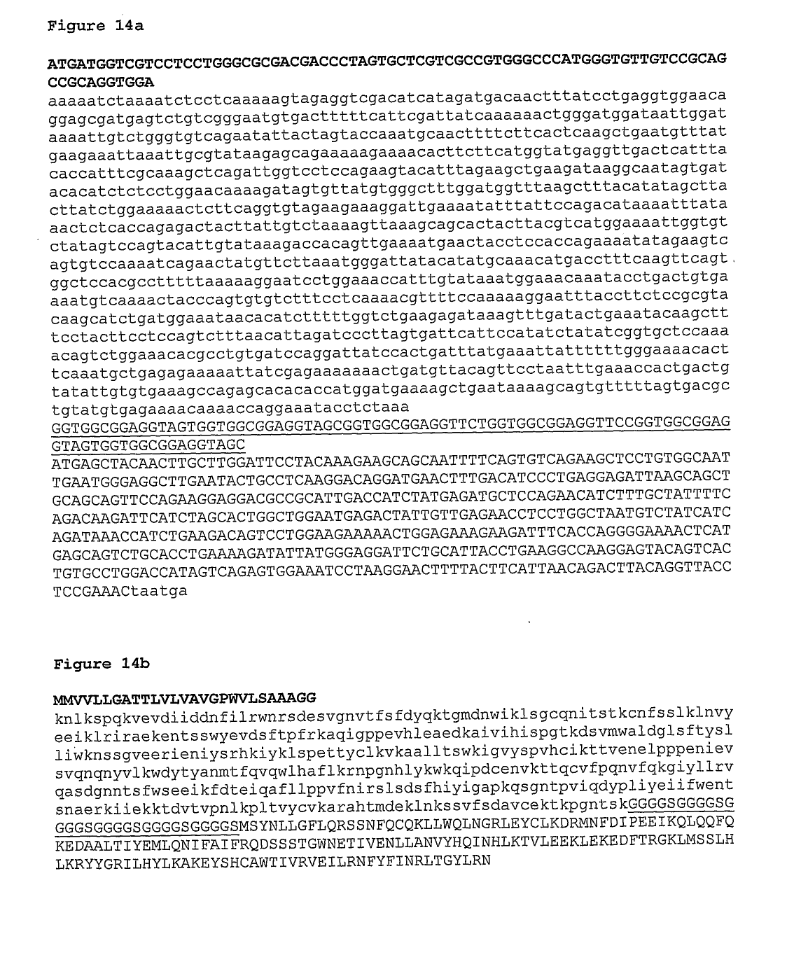

FIG. 13a is the nucleic acid sequence of LR 7B1; FIG. 13b is the amino acid sequence of LR 7B1;

FIG. 14a is the nucleic acid sequence of LR 7C1; FIG. 14b is the amino acid sequence of LR 7C1;

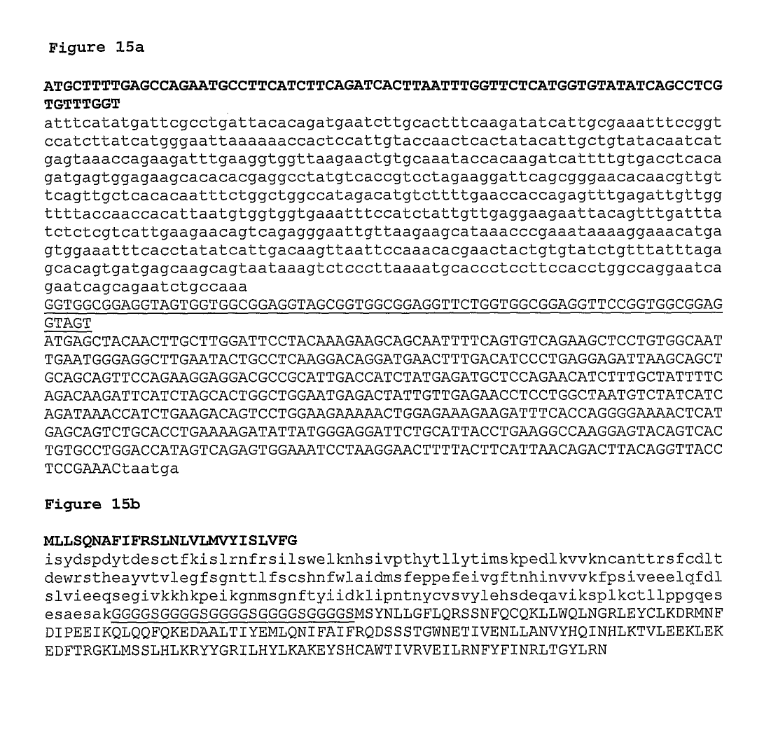

FIG. 15a is the nucleic acid sequence of LR 7D1; FIG. 15b is the amino acid sequence of LR 7D1;

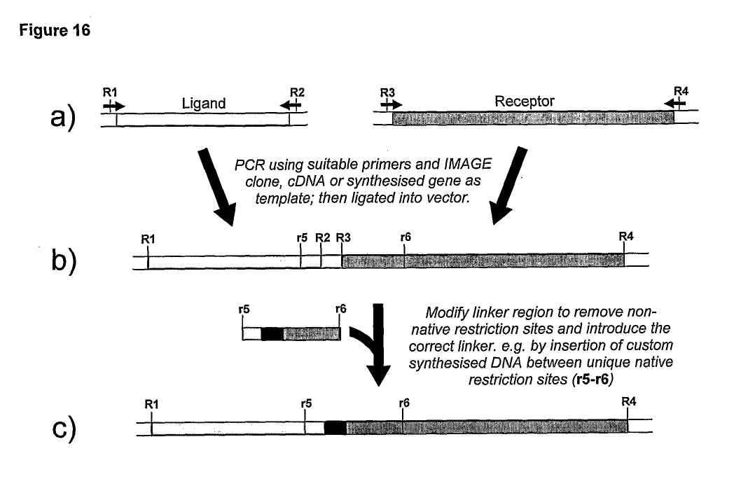

FIG. 16 a) PCR was used to generate DNA consisting of the gene of interest flanked by suitable restriction sites (contained within primers R1-4). b) The PCR products were ligated into a suitable vector either side of the linker region. c) The construct was then modified to introduce the correct linker, which did not contain any unwanted sequence (i.e. the non-native restriction sites);

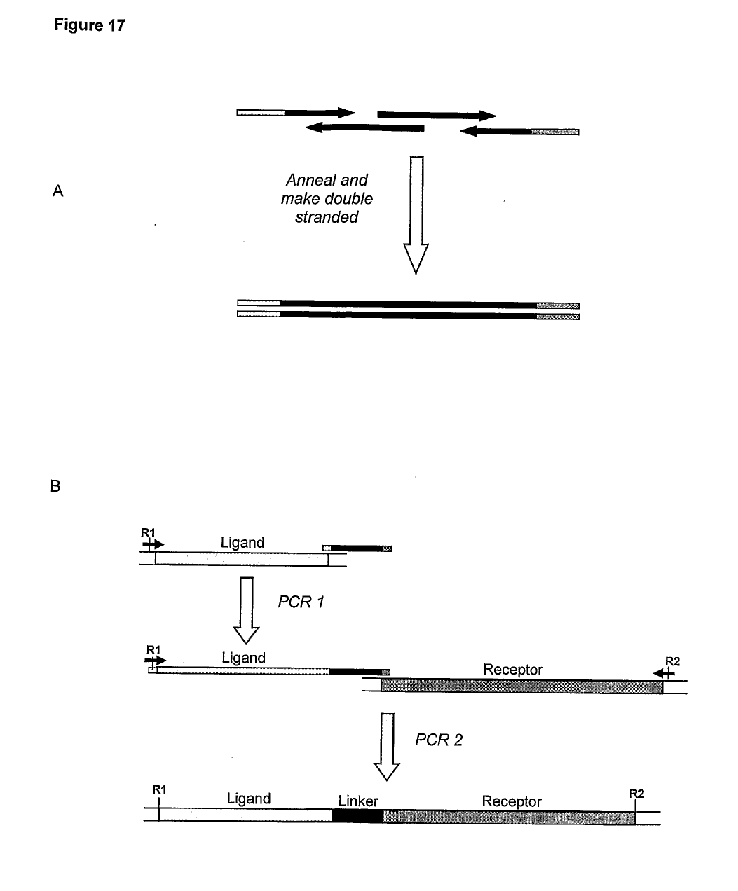

FIG. 17 a) Oligonucleotides were designed to form partially double-stranded regions with unique overlaps and, when annealed and processed would encode the linker with flanking regions which would anneal to the ligand and receptor. b) PCRs were performed using the “megaprimer” and terminal primers (R1 and R2) to produce the LR-fusion gene. The R1 and R2 primers were designed so as to introduce useful flanking restriction sites for ligation into the target vector;

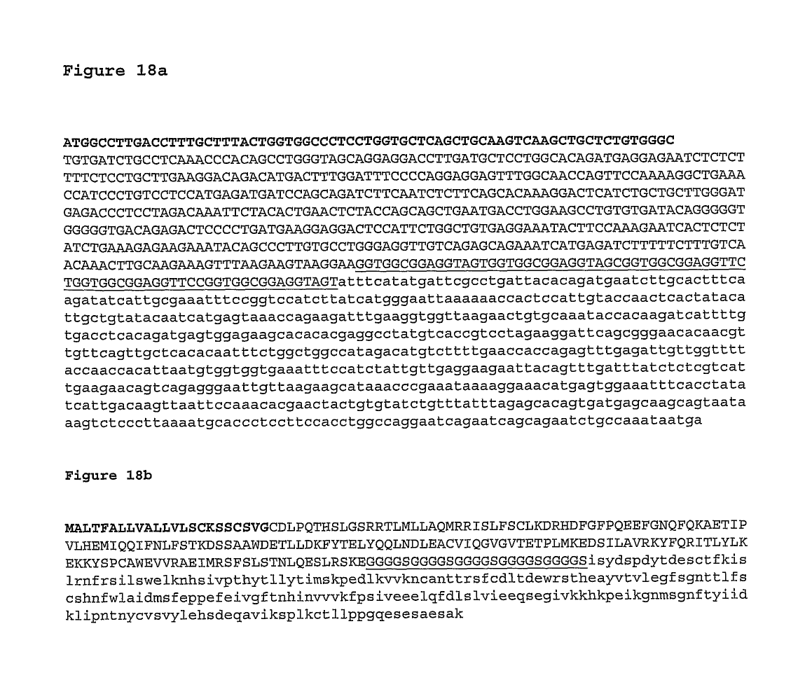

FIG. 18a is the nucleic acid sequence of LR a7B1; FIG. 18b is the amino acid sequence of LR a7B1;

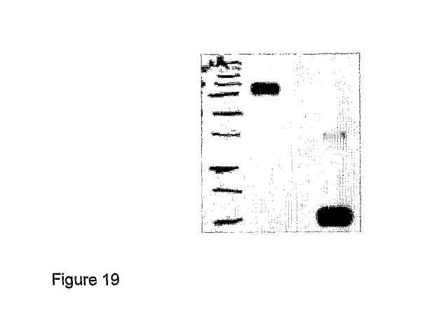

FIG. 19 illustrates a western blot of CHO cell expressed a7B1. Samples were prepared as described in the presence of DTT. Lane 1: Ladder, Lane 2: a7B1 (10× concentrated media from stable cell line), Lane 3: GAP, Lane 4: Positive control, 250 ng rh-IFNalpha2B. A7B1 separates as a distinct band of approximate MW 75-100 kDa: Non glycosylated MW=45.5 kDa. IFN control has a MW of 19.2 kDa;

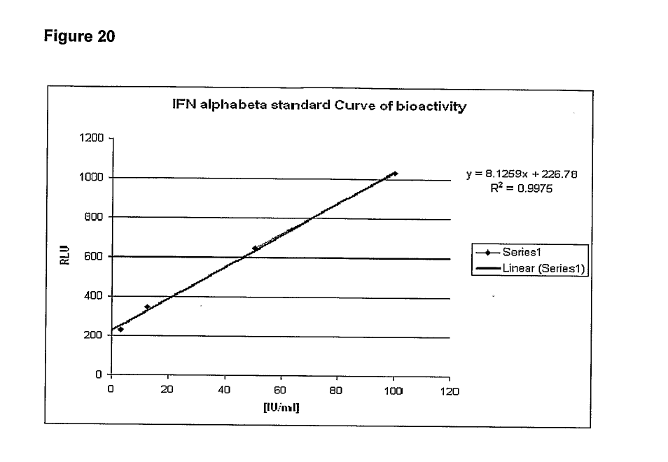

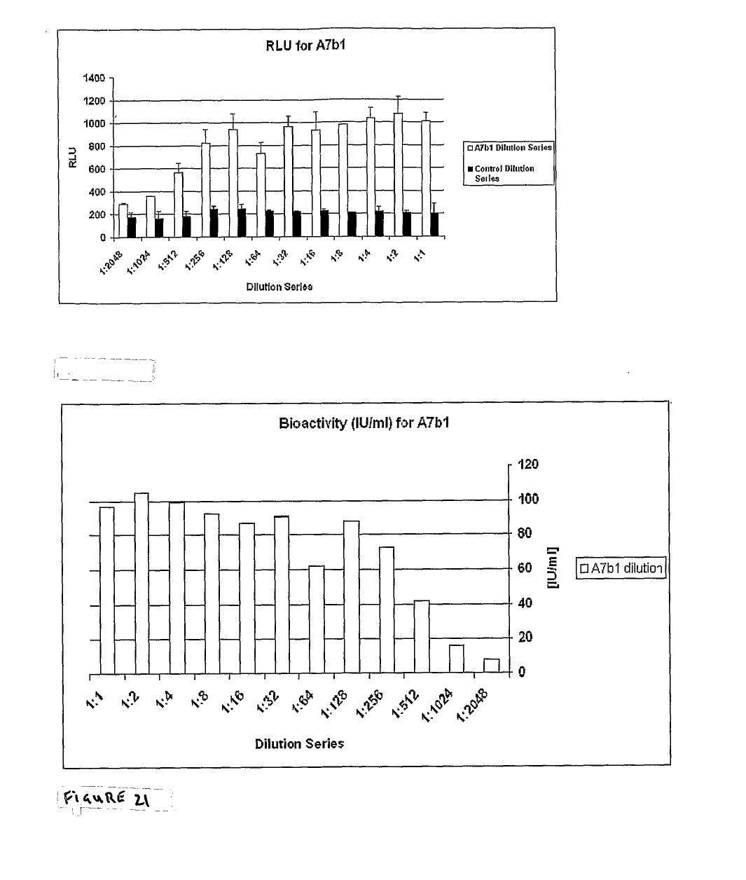

FIG. 20 illustrates the biological activity of interferon α 2b; and

FIG. 21 illustrates the biological activity of interferon α 2b chimera A7B1.

Materials and Methods

Interferon Bioassay

Commercially available bioassays can be used to test interferon, see http://www.sbhsciences.com/index.asp, SBH Sciences Inc and http://www.biocompare.com/index.asp, Biocompare Inc. In addition methods that assay the activity of interferon are described in Lleonart et al (Nature Biotech (1990) 8: 1263-1267; Sedmk and Grossberg (J. Gen Virology (1973) 21: 1-7; and Baumgarth and Kelso (J of Virology (1996) 70(7): p 4411-4418.

Immunological Testing

Immunoassays that measure the binding of ligand or receptor to polyclonal and monoclonal antibodies are known in the art. Commercially available antibodies are available to detect the ligand or receptor in samples and also for use in competitive inhibition studies. For example see http://www.abcam.com/index.html, Abcam PLC.

Recombinant Production of Fusion Proteins

The components of the fusion proteins were generated by PCR using primers designed to anneal to the ligand or receptor and to introduce suitable restriction sites for cloning into the target vector (FIG. 16a). The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector (FIG. 16b). The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification (FIG. 16c).

Alternatively, the linker with flanking sequence, designed to anneal to the ligand or receptor domains of choice, was initially synthesised by creating an oligonucleotide duplex and this processed to generate double-stranded DNA (FIG. 17a). PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice (FIG. 17b).

Expression and Purification of Fusion Proteins

Expression was carried out in a suitable system (e.g. mammalian CHO cells, E. coli) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the RL-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

Characterisation of LR-Fusions

Denaturing PAGE, native PAGE gels and western blotting were used to analyse the fusion polypeptides and western blotting performed with antibodies non-conformationally sensitive to the LR-fusion. Native solution state molecular weight information can be obtained from techniques such as size exclusion chrmoatography using a Superose G200 analytical column and analytical ultracentrifugation.

Construction of Chimeric Clones

All clones were ligated using the restriction enzymes Nhe1/HindIII, into the mammalian expression plasmid pSecTag-link. Clones were attached to the secretion signal for human interferon for efficient secretion into cell media. The whole gene for a7B1 [FIG. 18a] was cloned using gene synthesis and cloned into the mammalian expression vector pSecTag-link to form pIFNsecTag-a7B1

Mammalian Expression of IFN Chimeric Clones

A mammalian expression system has been established using a modification of the invitrogen vector pSecTag-V5/FRT-Hist

Invitrogen's FIp-In System

This system allows for the rapid generation of stable clones into specific sites within the host genome for high expression. This can be used with either secreted or cytoplasmic expressed proteins. FIp-In host cell lines (fIp-In CHO) have a single FIp recombinase target (FRT) site located at a transcriptionally active genomic locus

Stable cell lines are generated by co-transfection of vector (Containing FRT target site) and pOG44 (a [plasmid that transiently expresses fIp recombinase) into FIp-In cell line. Selection is with Hygromycin B. There is no need for clonal selection since integration of DNA is directed.

Culturing FIp-In Cell lines: followed manufactures instruction using basic cell culture techniques.

Stable Transfection of CHO FIp-In Cells using Fugene-6

The day before transfection CHO FIp-In cells were seeded at 6×10E5 per 100 mm petri dish in a total volume of 10 ml of Hams F12 media containing 10% (v/v) Fetal Calf Serum, 1% Penicillin/streptomycin and 4 mM L-glutamine. The next day added 570 μl of serum free media (containing no antibiotics) to a 1.5 ml polypropylene tube. 30 μl of fugene-6 was then added and mixed by gentle rolling. A separate mix of plasmids was set up for each transfection which combined 2 μg plasmid of interest with 18 μg pOG44 (plasmid contains recombinase enzyme necessary for correct integration of plasmid into host genome). Control plate received no plasmid. This was mixed with fugene-6 by gentle rolling, left @ RmT for 15 minutes, then applied drop-wise to the surface of the each petri dish containing CHO FIp-In cells in F12 media+10% FCS. The plates were gently rolled to ensure good mixing and left for 24 hrs @ 37° C./5% CO2. The next day media was exchanged for selective media containing hygromycin B @ 600 ug/ml. Cells were routinely kept at 60% confluency or less. Cells were left to grow in the presence of 600 ug/ml hygromycin B until control plate cells (non transfected cells) had died (i.e. no hygromycin resistance).

SDS-PAGE Analysis

Testing Expression from Stable CHO Cell Lines

Confluent CHO FIp-In cell lines expressing the protein of interest were grown in 75 cm2 flasks for approximately 3-4 days in serum free media, at which point samples were taken and concentrated using acetone precipitation. Samples were mixed with an equal volume of laemmli loading buffer in the presence or absence of 25 mM DTT and boiled for 5 minutes. Samples were analysed by SDS-PAGE and transferred to a PVDF membrane. After blocking in 5% (w/v) Milk protein in PBS-0.05% (v/v) Tween 20, sample detection was carried out using a specific anti-IGF-1 antibody together with a Horse Radish Peroxidase (HRP) conjugated secondary antibody. Visualisation was by chemiluminesence on photographic film using an HRP detection kit.

Testing Expression from Transient Tansfections of CHO fIpIn

CHO FIp-In cells were seeded at 0.25×10E6 cells per well of a 6 well plate in a total volume of 2 ml media (DMEM, F12, 10% FCS+P/S+L-glutamine+Zeocin). Cells were left to grow o/n. Cells were then transfected using either TransIT-CHO Reagent (Mirus) or fugene-6 at the specified reagent ratios stated in table 1. Control transfections were set up using 1B7stop (GH containing chimeric molecule). Briefly, if using TransIT reagent, 200 ul of Serum free media (OPTI MEM) was added to a 1.5 ml eppendorff per transfection followed by 2 ug DNA. The tubes were left for 15 minutes at RmT. 1 ul of CHO Mojo Reagent was then added, mixed and left for a further 15 minutes. Media was changed to serum free and the transfection mix pippetted dropwise onto the surface of the appropriate well. Briefly, if using Fugene-6 reagent, 94 ul of Serum free media (OPTI MEM) was added to a 1.5 ml eppendorff per transfection followed by 2 ug DNA. The tubes were left for 15 minutes at RmT. Trasfection mix was then pippetted drop wise onto the surface of the appropriate well containing serum free media. All plate were left @ 37° C./5% CO2 for 2-3 days

| | | | Transfection 1 | | Transfection | Transfection |
|---|---|---|---|---|---|---|
| | | Volume | CHO Mojo | | 2 | 3 |
| | | containing | Reagent | | Fugene-6 to | Fugene-6 to |
| | | 2ug of | (5ug/ul | TransIT-CHO | DNA ratio = | DNA ratio = |
| Plasmid | mg/ml | DNA | stock) | Reagent (A) | 3:2 | 3:1 |
| A7b1 | 1.6 | 1.25ul | 1 ul | 4ul | — | — |
| 1B7 Stop | 1 | 2ul | 1 ul | 4ul | 3ul | 6ul |

Statistics

Two groups were compared with a Student's test if their variance was normally distributed or by a Student-Satterthwaite's test if not normally distributed. Distribution was tested with an F test. One-way ANOVA was used to compare the means of 3 or more groups and if the level of significance was p<0.05 individual comparisons were performed with Dunnett's tests. All statistical tests were two-sided at the 5% level of significance and no imputation was made for missing values.

Interferon Bioassay

A stable CHO FIpIn cell line expressing the soluble Interferon Alpha Chimeric protein: A7b1 (AS-80) was grown by ARCBioserv. Control media consisting of non transfected CHO cells were also grown at the same time and treated in the same way. The Bioactivity of each sample was detected using a Human Type I Interferon Activity Detection kit (Neutekbio iLite AlphaBeta Kit: Galway, Ireland, catalogue #46-88R, Lot #0810601). Both media were concentrated and filter sterilised prior to undertaking serial dilution of sample. Manufacturers instructions for kit usage were followed throughout.

The results clearly show that A7b1 has bioactivity and that controls show no bioactivity.

The dilutions from 1:1 to 1:128 have reached the maximum RLU for the assay see FIGS. 20 and 21. Further dilutions show a good dose response curve of Bioactivity: This relates to a IU/ml range of ˜16-20,000.

1. A nucleic acid molecule comprising a nucleic acid sequence that encodes a polypeptide that has the activity of interferon α 2b wherein said polypeptide comprises interferon α 2b or part thereof linked, directly or indirectly, to the interferon binding domain of an interferon receptor.

2. A fusion polypeptide comprising: the amino acid sequence of an interferon α 2b, or active binding part thereof, linked, directly or indirectly, to the binding domain of an interferon receptor.

3. A fusion polypeptide according to claim 2 wherein said fusion polypeptide comprises SEQ ID NO: 34 or 35 or an amino acid sequence that is at least 75% identical to the amino acid sequence represented in SEQ ID NO: 34 or 35 over all or part of its length.

4. A fusion polypeptide according to claim 2 wherein said fusion polypeptide comprises SEQ ID NO: 34 or 35 or an amino acid sequence that is at least 80% identical to the amino acid sequence represented in SEQ ID NO: 34 or 35 over all or part of its length.0

5. A fusion polypeptide according to claim 2 wherein said fusion polypeptide comprises SEQ ID NO: 34 or 35 or an amino acid sequence that is at least 85% identical to the amino acid sequence represented in SEQ ID NO: 34 or 35 over all or part of its length.

6. A fusion polypeptide according to claim 2 wherein said fusion polypeptide comprises SEQ ID NO: 34 or 35 or an amino acid sequence that is at least 90% identical to the amino acid sequence represented in SEQ ID NO: 34 or 35 over all or part of its length.

7. A fusion polypeptide according to claim 2 wherein said fusion polypeptide comprises SEQ ID NO: 34 or 35 or an amino acid sequence that is at least 95% identical to the amino acid sequence represented in SEQ ID NO: 34or 35 over all or part of its length.

8. A fusion polypeptide according to claim 2 wherein said fusion polypeptide comprises SEQ ID NO: 34 or 35.

9. A fusion polypeptide according to any of claims 2-8 wherein said fusion polypeptide comprises at least one fibronectin III binding domain.

10. A fusion polypeptide according to claim 9 wherein said fusion polypeptide comprises two fibronectin III binding domains.

11. A fusion polypeptide according to claim 9 wherein said fusion polypeptide comprises three fibronectin III binding domains.

12. A fusion polypeptide according to claim 9 wherein said fusion polypeptide comprises amino acid residues 28-436 of SEQ ID NO: 5.

13. A fusion polypeptide according to claim 9 wherein said fusion polypeptide comprises amino acid residues 27-243 of SEQ ID NO: 6.

14. A fusion polypeptide according to any of claims 2-13 wherein interferon α 2b is linked to an interferon binding domain of an interferon receptor wherein said interferon α 2b is positioned amino terminal to said binding domain in said fusion polypeptide.

15. A fusion polypeptide according to any of claims 2-13 wherein interferon α 2b is linked to an interferon binding domain of an interferon receptor wherein said interferon α 2b is positioned carboxyl-terminal to said binding domain in said fusion polypeptide.

16. A fusion polypeptide according to any of claims 2-15 wherein said interferon α 2b is linked to the binding domain of the of the interferon receptor by a peptide linker.

17. A fusion polypeptide according to claim 16 wherein said peptide linking molecule comprises at least one copy of the peptide Gly Gly Gly Gly Ser.

18. A fusion polypeptide according to claim 17 wherein said peptide linking molecule comprises 2, 3, 4, 5 or 6 copies of the peptide Gly Gly Gly Gly Ser.

19. A fusion polypeptide according to claim 18 wherein said peptide linking molecule consists of 5 copies of the peptide Gly Gly Gly Gly Ser.

20. A fusion polypeptide according to any of claims 2-15 wherein said polypeptide does not comprise a peptide linking molecule and is a direct fusion of interferon α 2b and the interferon binding domain of the interferon receptor.

21. A homodimer consisting of two polypeptides wherein each of said polypeptides comprises:
i) a first part comprising interferon α 2b, or a receptor binding domain thereof, optionally linked by a peptide linking molecule to
ii) a second part comprising at least one interferon binding domain or part thereof, of an interferon receptor.

22. A homodimer according to claim 21 wherein said homodimer comprises two polypeptides comprising SEQ ID NO: 34 or 35.

23. A vector comprising a nucleic acid molecule according to claim 1.

24. A cell transfected or transformed with a nucleic acid molecule or vector according to claim 1 or 23.

25. A cell according to claim 24 wherein said cell is a eukaryotic cell.

26. A cell according to claim 24 wherein said cell is a prokaryotic cell.

27. A pharmaceutical composition comprising a polypeptide according to any of claims 2-20 including an excipient or carrier.

28. A pharmaceutical composition according to claim 27 wherein said composition is combined with a further therapeutic agent.

29. A method to treat a human subject suffering from cancer comprising administering an effective amount of a polypeptide according to any of claims 2-20.

30. A method according to claim 29 wherein said cancer is melanoma.

31. A method according to claim 29 or 30 wherein said polypeptide is administered at two day intervals.

32. A method according to claim 29 or 30 wherein said polypeptide is administered at weekly intervals.

33. A method according to claim 29 or 30 wherein said polypeptide is administered at 2 weekly intervals.

34. A method according to claim 29 or 30 wherein said polypeptide is administered at monthly intervals.

35. The use of a polypeptide according to any of claims 2-20 for the manufacture of a medicament for the treatment of cancer.

36. Use according to claim 35 wherein said cancer is melanoma.

37. A monoclonal antibody that binds the polypeptide or dimer according to any of claims 2-20.

38. A monoclonal antibody according to claim 37 wherein said antibody is an antibody that binds the polypeptide or dimer but does not specifically bind interferon or interferon receptor individually.

39. A method for preparing a hybridoma cell-line producing monoclonal antibodies according to the invention comprising the steps of:
i) immunising an immunocompetent mammal with an immunogen comprising at least one polypeptide according to any of claims 2-20;
ii) fusing lymphocytes of the immunised immunocompetent mammal with myeloma cells to form hybridoma cells;
iii) screening monoclonal antibodies produced by the hybridoma cells of step (ii) for binding activity to the polypeptide of (i);
iv) culturing the hybridoma cells to proliferate and/or to secrete said monoclonal antibody; and
v) recovering the monoclonal antibody from the culture supernatant.

40. A method according to claim 39 wherein said immunocompetent mammal is a mouse or rat.

41. A hybridoma cell-line obtained or obtainable by the method according to claim 39 or 40.

42. A diagnostic test to detect a polypeptide according to any of claims 2-20 in a biological sample comprising:
i) providing an isolated sample to be tested;
ii) contacting said sample with a ligand that binds the polypeptide or dimer according to the invention; and
iii) detecting the binding of said ligand in said sample.

43. A test according to claim 42 wherein said ligand is an antibody; preferably a monoclonal antibody.


SEQUENCE LISTING (36 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 3 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 4 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 5 - protein, 557 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 6 - protein, 515 aa, Homo sapiens [sequence omitted]

SEQ ID NO: 7 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 8 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 9 - protein, 489 aa, Homo sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 10 - protein, 472 aa, Homo sapiens
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 11 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 12 - protein, 181 aa, Homo sapiens
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 13 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 14 - protein, 175 aa, Homo sapiens
VPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 15 - protein, 200 aa, Homo sapiens
MKLDMTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 16 - protein, 175 aa, Homo sapiens
VPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 17 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 18 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 19 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 20 - protein, 500 aa, Homo sapiens
RPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 21 - DNA, 1884 bp, Artificial
  nucleotide sequence encoding interferon/interferon receptor fusion protein
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacggtggcggaggtagtggtggcggaggtagcggtggcggaggttctggtggcggaggttccggtggcggaggtagtggtggcggaggtagcaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttacttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaataatga

SEQ ID NO: 22 - protein, 626 aa, Artificial
  interferon/interferon receptor fusion protein
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLLIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 23 - protein, 605 aa, Artificial
  interferon/interferon receptor fusion protein
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSGGGGSGGGGSGGGGSKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLLIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 24 - DNA, 1293 bp, Artificial
  nucleotide sequence encoding interferon/interferon receptor fusion protein
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacggtggcggaggtagtggtggcggaggtagcggtggcggaggttctggtggcggaggttccggtggcggaggtagtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtggtggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccaaataatga

SEQ ID NO: 25 - protein, 429 aa, Artificial
  interferon/interferon receptor fusion protein
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSGGGGSGGGGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 26 - protein, 408 aa, Artificial
  interferon/interferon receptor fusion protein
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSGGGGSGGGGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 27 - DNA, 1902 bp, Artificial
  nucleotide sequence encoding interferon/interferon receptor fusion protein
atgatggtcgtcctcctgggcgcgacgaccctagtgctcgtcgccgtgggcccatgggtgttgtccgcagccgcaggtggaaaaaatctaaaatctcctcaaaaagtagaggtcgacatcatagatgacaactttatcctgaggtggaacaggagcgatgagtctgtcgggaatgtgactttttcattcgattatcaaaaaactgggatggataattggataaaattgtctgggtgtcagaatattactagtaccaaatgcaacttttcttcactcaagctgaatgtttatgaagaaattaaattgcgtataagagcagaaaaagaaaacacttcttcatggtatgaggttgactcatttacaccatttcgcaaagctcagattggtcctccagaagtacatttagaagctgaagataaggcaatagtgatacacatctctcctggaacaaaagatagtgttatgtgggctttggatggtttaagctttacatatagcttacttatctggaaaaactcttcaggtgtagaagaaaggattgaaaatatttattccagacataaaatttataaactctcaccagagactacttattgtctaaaagttaaagcagcactacttacgtcatggaaaattggtgtctatagtccagtacattgtataaagaccacagttgaaaatgaactacctccaccagaaaatatagaagtcagtgtccaaaatcagaactatgttcttaaatgggattatacatatgcaaacatgacctttcaagttcagtggctccacgcctttttaaaaaggaatcctggaaaccatttgtataaatggaaacaaatacctgactgtgaaaatgtcaaaactacccagtgtgtctttcctcaaaacgttttccaaaaaggaatttaccttctccgcgtacaagcatctgatggaaataacacatctttttggtctgaagagataaagtttgatactgaaatacaagctttcctacttcctccagtctttaacattagatcccttagtgattcattccatatctatatcggtgctccaaaacagtctggaaacacgcctgtgatccaggattatccactgatttatgaaattattttttgggaaaacacttcaaatgctgagagaaaaattatcgagaaaaaaactgatgttacagttcctaatttgaaaccactgactgtatattgtgtgaaagccagagcacacaccatggatgaaaagctgaataaaagcagtgtttttagtgacgctgtatgtgagaaaacaaaaccaggaaatacctctaaaggtggcggaggtagtggtggcggaggtagcggtggcggaggttctggtggcggaggttccggtggcggaggtagtggtggcggaggtagcatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaatga

SEQ ID NO: 28 - protein, 632 aa, Artificial
  interferon/interferon receptor fusion protein
MMVVLLGATTLVLVAVGPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLLIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKGGGGSGGGGSGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 29 - protein, 605 aa, Artificial
  interferon/interferon recptor fusion protein
KNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLLIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKGGGGSGGGGSGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 30 - DNA, 1308 bp, Artificial
  nucleotide sequence encoding interferon/interferon receptor fusion protein
atgcttttgagccagaatgccttcatcttcagatcacttaatttggttctcatggtgtatatcagcctcgtgtttggtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtggtggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccaaaggtggcggaggtagtggtggcggaggtagcggtggcggaggttctggtggcggaggttccggtggcggaggtagtatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactaatga

SEQ ID NO: 31 - protein, 434 aa, Artificial
  interferon/interferon receptor fusion protein
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAKGGGGSGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 32 - protein, 408 aa, Artificial
  interferon/interferon receptor fusion protein
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAKGGGGSGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 33 - DNA, 1296 bp, artificial
  interferon fusion protein DNA sequence
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaaggtggcggaggtagtggtggcggaggtagcggtggcggaggttctggtggcggaggttccggtggcggaggtagtatttcatatgattcgcctgattacacagatgaatcttgcactttcaagatatcattgcgaaatttccggtccatcttatcatgggaattaaaaaaccactccattgtaccaactcactatacattgctgtatacaatcatgagtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacattaatgtggtggtgaaatttccatctattgttgaggaagaattacagtttgatttatctctcgtcattgaagaacagtcagagggaattgttaagaagcataaacccgaaataaaaggaaacatgagtggaaatttcacctatatcattgacaagttaattccaaacacgaactactgtgtatctgtttatttagagcacagtgatgagcaagcagtaataaagtctcccttaaaatgcaccctccttccacctggccaggaatcagaatcagcagaatctgccaaataatga

SEQ ID NO: 34 - protein, 430 aa, artificial
  interferon alpha 2b fusion protein
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSGGGGSGGGGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 35 - protein, 407 aa, Artificial
  interferon 2b fusion protein processed
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSGGGGSGGGGSISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVVVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 36 - protein, 5 aa, Artificial
  peptide linking molecule
GGGGS